(19) United States
(12) Patent Application Publication
(10) Pub. No.: US 2005/0016537 A1
(43) Pub. Date: Jan. 27, 2005

(54) Second-stage regulator for scuba divers

(75) Inventor: Stefano Pedemonte, Ceranesi (IT)

Correspondence Address: Grant E. Pollack, Esq.;Steinberg & Raskin, P.C., 15th Floor, 1140 Avenue of the Americas, New York, NY, 10036-5803, US

(73) Assignee: Cressi-Sub S.p.A.

(21) Appl. No.: 10/899,198

(22) Filed: Jul. 26, 2004

(30) Foreign Application Priority Data
Jul. 25, 2003 (IT) FI2003A000199

(51) Int. Cl. A62B 7/04; A61M 16/00

(52) U.S. Cl. 128/204.26

(57) ABSTRACT

A second-stage regulator for scuba divers, wherein the user's inhalation effort is lessened considerably by the reduction of the friction between selected components thereof. Coaxially to the regulator poppet, a flexible sleeve is sealingly connected to the poppet and the baffle, so as to avoid blow-by of gaseous mixture through the baffle opening through which there extends the tail of the poppet connected to the lever of the regulator extending in the outlet chamber thereof. The poppet head is placed in ferrule with an at least part-circular profile abutting the inner part of the intermediate chamber to allow the poppet oscillation. The lever end contacting the diaphragm, separating the outlet chamber from the outside, has a generally arched shape with a profile such that the length of the arch between two adjacent contact points measured along the lever is generally equal to the length of the segment between the same adjacent contact points measured along the diaphragm.

### FIELD OF THE INVENTION

The present invention relates generally to diving equipment and more particularly refers to an improved second-stage regulator for scuba diver. More precisely, the invention concerns an improvement to a regulator constituting the second pressure-reducing stage in a device for delivering air, or a mixture of air and oxygen, to the scuba diver's mouthpiece.

### BACKGROUND ART

It is known that the supply of air, or of the air-oxygen mixture, which is fed to the mouthpiece of the scuba diver from a high-pressure tank, passes via a primary pressure-reducing regulator to a second-stage regulator which supplies the mixture to the mouthpiece of the scuba diver when pressure within the regulator is diminished by a diver's inhalation.

Second-stage regulators of the known type have an inlet chamber connected to the outlet of the first-stage regulator, and an outlet chamber connected to the mouthpiece of the user and separated from the outside environment by an elastically deformable diaphragm. The diaphragm is connected via a lever to a poppet which closes off the passage between the two chambers.

The pressure inside the inlet chamber is kept constant at approximately ten bars as the pressure in the tank varies thanks to appropriate calibration of the first-stage regulator.

When the user does not breathe, his or her lungs, the mouthpiece, the outlet chamber and the outside environment are at the same pressure.

When the user inhales, a vacuum is created inside the outlet chamber and the diaphragm bends towards the interior of said chamber, moving the poppet, which normally closes the passage between the inlet chamber and the outlet chamber, towards its opening position.

The opening of the passage between the inlet chamber and outlet chamber creates an overpressure in the outlet chamber, so that the diaphragm returns into the rest position, moving the lever and returning the poppet into the starting position wherein the passage between the inlet chamber and the outlet chamber is closed once again.

The movement of these mechanical actuating members, i.e. the diaphragm, the lever and the poppet, is consequently controlled by the vacuum produced by the user when he inhales and the energy required must also allow for the energy dissipated by friction between these interconnected mechanical members. For a better understanding of the various causes of friction occurring in a second-stage regulator of known type, it is useful to examine its structure in detail, referring to the attached FIGS. 1-4.

FIG. 1 shows a second-stage regulator of known type, generically identified by the letter D, which comprises an inlet chamber D1, which is always at the first-stage regulator's outlet pressure, an intermediate chamber D2 and an outlet chamber D3 connected to the user's mouthpiece D4. The inlet chamber D1 is separated from the intermediate chamber D2 by a valve seat D5 supporting the seal D6 on the head D7 of a poppet D8, whose tail D9 passes loosely through the hole D10a in a baffle D10 placed between the intermediate chamber D2 and the outlet chamber D3.

The purpose of the baffle D10 is to support a spring D11 that compresses the head of the poppet D8 against the valve seat D5.

The outlet chamber D3 is separated from the outside environment D12 by a diaphragm D13. Against a thin rigid plate D14 on the inner surface of the diaphragm D13, there rests the outer end D15 of a lever D16 whose other, inner end D17 is hinged to the baffle D10 and supports the tail D9 of the poppet D8 projecting from the baffle D10 into the outlet chamber D3.

It is known that, under balanced conditions, the outlet chamber D3 of the second-stage regulator is at the same pressure as the user's lungs, which are at the same pressure as the outside environment D12. When the user inhales, a vacuum is created in the outlet chamber D3 with respect to the outside environment D12 and this causes the diaphragm D13 to flex inwards, with a consequent rotation of the lever D16 in the direction of the arrow F1 (indicated by a dotted line in FIG. 2) and a displacement of the poppet D8 in the direction of the arrow F2, with the consequent passage of breathable gas mixture under pressure from the inlet chamber D1 to the outlet chamber D3, through the intermediate chamber D2 connected to the outlet chamber D3 by a wide passage D18.

As soon as the mixture coming from the inlet chamber D1 reaches the outlet chamber D3, thereby increasing the pressure in the latter, the diaphragm D13 returns to its rest position, and so do the lever D16 and the poppet D8, which closes the seat D5 once again, separating the inlet chamber D1 from the intermediate chamber D2 and from the outlet chamber D3 until the user inhales again.

In an ideal second-stage regulator, the vacuum created by the user inhaling should be minimal in order to facilitate his unavoidable respiratory effort. As mentioned earlier, however, the vacuum that the user produces by inhaling must also cope with the unavoidable friction accompanying the movement of the diaphragm D13, the lever D16 and the poppet D8. Moreover, said vacuum cannot be reduced by increasing the dimensions of the diaphragm D13 because the size of the second-stage regulator must be limited in relation to the apparatus connected upstream and downstream of the regulator.

A first cause of friction is due to the blow-by of the gas mixture from the intermediate chamber D2 to the outlet chamber D3, through the annular opening between the tail D9 of the poppet D5 and the hole D10a in the baffle D10. Although the majority of the breathable gas mixture passes from the intermediate chamber D2 to the outlet chamber D3 through the wide passage D18, a modest quantity nonetheless inevitably also passes through the above-mentioned annular opening and, since the passage of the mixture from the intermediate chamber D2 to the outlet chamber D3 is accompanied by expansion, and consequently also cooling, the humidity contained in the mixture is converted into tiny ice crystals that generate friction during the axial movement of the tail D9 of the poppet D8.

A second cause of friction is due to rubbing of the outer end D15 of the lever D16 against the inner surface of the plate D14 applied under the diaphragm D13, as the latter flexes into the outlet chamber D3 under the effect of the vacuum induced by the user inhaling. Despite the generally curved shape of the outer end D15 of the lever D16, the point of contact between lever and diaphragm varies as the latter flexes, thereby creating a sliding friction that has to be overcome by part of the vacuum generated by the user.

A third cause of friction is due to rubbing of the inner end D17 of the lever D16 where it comes into contact with the tail D9 of the poppet D8.

As shown in FIGS. 2, 3 and 4, the inner end of the lever D16 usually comprises a first flange D19, substantially perpendicular to the inner end D17 of the lever D16. At the end of flange D19 there is a second flange D20, substantially parallel to the inner end of the lever D16, and then a third flange D21 parallel to the first flange D19 and facing in the same direction. The profile of the inner end D17 of the lever D16 is consequently shaped substantially in the form of a Z by this succession of flanges D19, D20 and D21. Finally, the presence of a longitudinal slot D22 gives rise to the two branches of a Z-shaped fork that can fit around the tail D9 of the poppet D8 between a washer D23, supported by a nut D24 screwed onto the threaded end of the tail D9, and the side of the baffle D10 facing towards the outlet chamber D3.

The baffle D10 acts as a fulcrum for the third flange D21 of the inner end D17 of the lever D16 and, as it turns, it displaces the washer D23, together with the poppet D8, in the direction of the arrow F2, overcoming the force of the spring D1. As the lever D16 turns, the two branches of the third flange D21 inevitably slide against the washer D23 and the baffle D10 and the consequent friction has to be overcome by part of the vacuum generated by the user when he inhales.

### OBJECTS AND SUMMARY OF THE INVENTION

The general object of the present invention is to provide an improved second-stage regulator for scuba divers whose opening demands less effort from the user than known second-stage regulators, thereby facilitating the user's inhalatory action.

A particular object of the present invention is to provide an improved second-stage regulator for scuba divers of the above-mentioned type, wherein the friction due to the mechanical members is significantly reduced by comparison with the case of second-stage regulators of known type.

A further object of the present invention is to provide an improved second-stage regulator for scuba divers of the above-mentioned type, wherein the relative sliding of the various mutually contacting, mechanical members is eliminated and rolling friction essentially occurs.

An important characteristic of the second-stage regulator according to the present invention lies in that, inside the intermediate chamber and coaxial to the poppet, there is a flexible sleeve with an airtight connection to both the poppet and the baffle around said opening, thus preventing any blow-by of the gas mixture through the opening created by the tail of the poppet and the opening in the baffle containing said poppet, with the consequent formation of tiny ice crystals, which are one of the sources of friction and therefore of energy dissipation.

Another important characteristic of the second-stage regulator according to the present invention lies in that the head of the poppet is inside a ferrule of substantially rectangular cross section, whose section in the median longitudinal plane (which also includes the lever) has at least a part with a circular profile abutting against the inside wall of the intermediate chamber and enabling the poppet to oscillate in the longitudinal plane. In this way, the end of the lever attached to the tail of the poppet moves integrally with the tail, with negligible sliding, and any friction induced is only of the rolling type as the circular profile of the ferrule turns against the inner wall of the intermediate chamber.

Another important characteristic of the second-stage regulator according to the present invention lies in that the end of the lever in contact with the rigid plate associated with the diaphragm separating the outside environment from the regulator's outlet chamber has an arched shape following a profile such that the arch extending between two adjacent points of contact measured on the lever is equal to the length of the segment between the same adjacent points of contact measured on said rigid plate, so that the friction generated by the relative movement between the two members is substantially of the rolling type.

### BRIEF DESCRIPTION OF THE DRAWINGS

Further characteristics and advantages of the improved second-stage regulator according to the present invention will become apparent from the following description of one of its embodiments by way of a non-limiting example, with reference to the accompanying drawings, wherein:

FIG. 1 is a simplified longitudinal section of a second-stage regulator of known type;

FIG. 2 is an enlarged detail of the end portion of the actuating lever in the second-stage regulator of FIG. 1;

FIG. 3 is a longitudinal section taken along line III-III of FIG. 2;

FIG. 4 is a perspective view of the foot of the actuating lever in the regulator of FIG. 1;

FIG. 5 is a front perspective view of the second-stage regulator according to the invention;

FIG. 6 shows, on a larger scale, a longitudinal section taken along line VI-VI of FIG. 5;

FIG. 7 is an enlarged portion of FIG. 6;

FIG. 8 is an enlarged partial view of the actuating lever, controlled by the movement of the diaphragm;

FIG. 9 shows, on a larger scale, a section taken along line IX-IX of FIG. 6;

FIG. 10 is a side view of the second-stage regulator according to the invention;

FIG. 11 is a side view, wherein the regulator of FIG. 10 is shown with the cover protecting the diaphragm raised, with an exploded view of the diaphragm and its protection grid;

FIG. 12 is a perspective view of the same regulator showing the means for locking the cover to the regulator body;

FIG. 13 is a side view of the means for locking the regulator body's cover in the closed position;

FIG. 14 is an isometric view of the means for locking the cover of FIG. 12.

### DETAILED DESCRIPTION OF THE INVENTION

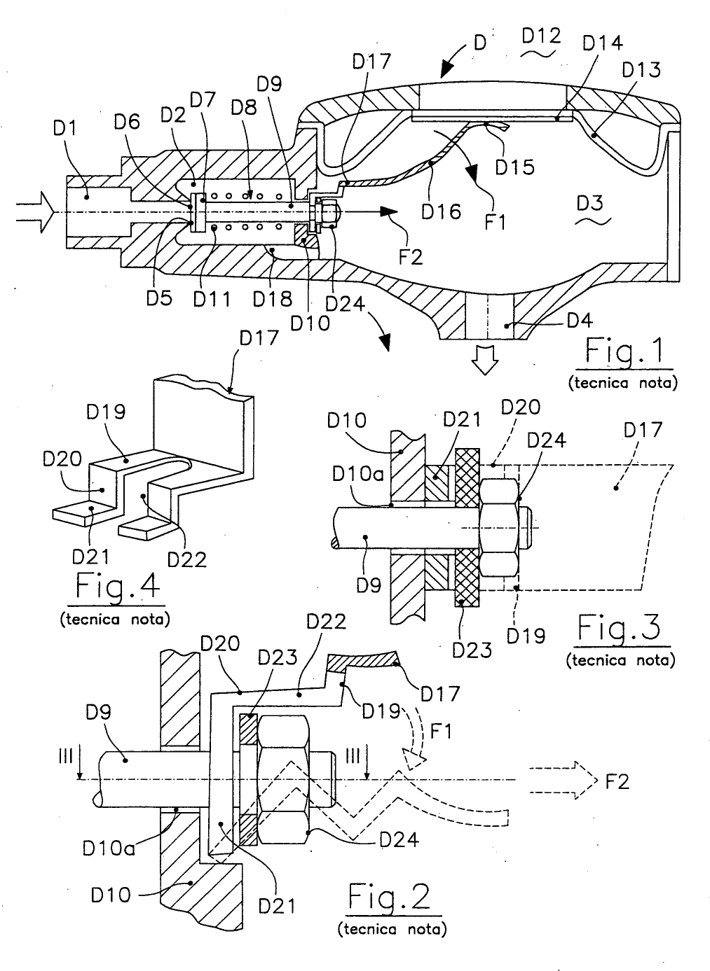

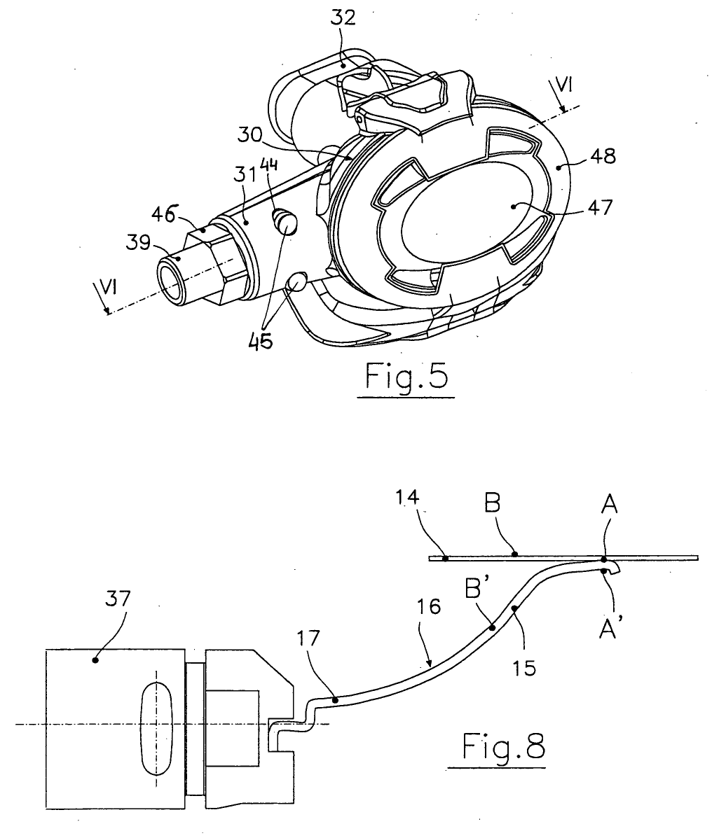

FIG. 5 and thereafter illustrate a preferred embodiment of the regulator according to the present invention. The reference numbers used in the figures are the same as those used in describing the second-stage regulator according to the known art, illustrated in FIGS. 1 to 4, except that the letter D is removed when describing similar structural elements. The components not contained in the second-stage regulator of known type are numbered starting from the reference numeral 30.

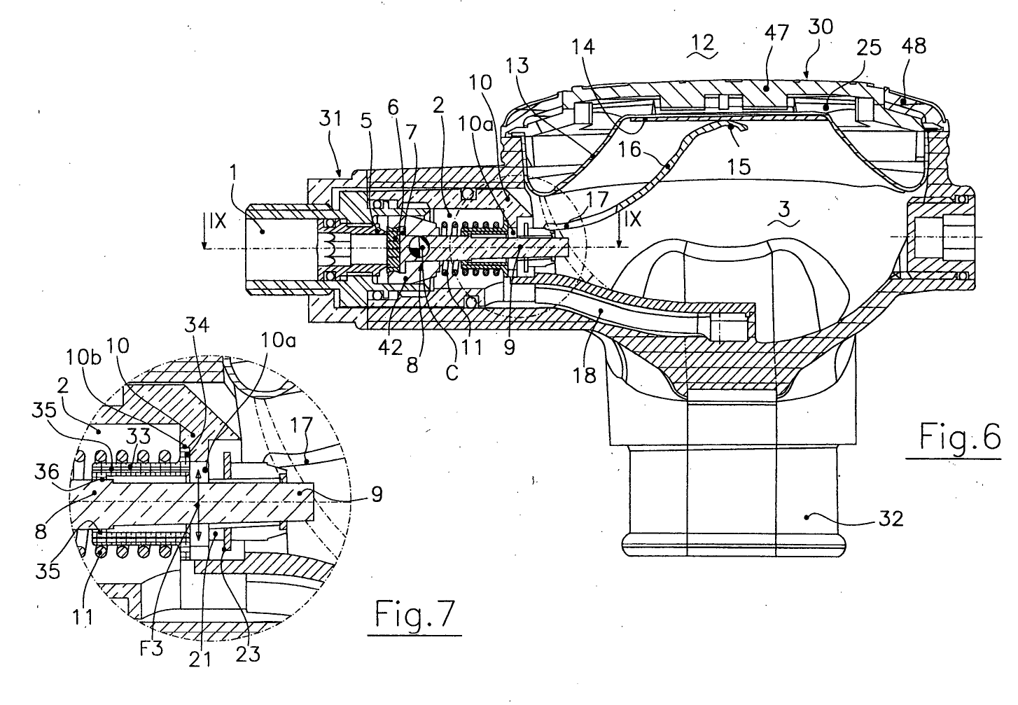

With reference to FIGS. 5 and 6, the numeral 30 is used to indicate a regulator body with an inlet conduit 31 and an outlet conduit 32. The inlet conduit 31 connects a first-stage regulator that delivers a breathable gas mixture at a constant pressure and its interior forms an inlet chamber 1 and an intermediate chamber 2, separated by a valve seat 5 supporting the seal 6 of the head 7 of a poppet 8. The tail 9 of the poppet 8 passes loosely through the hole 10a in a baffle 10, which separates the intermediate chamber 2 from an outlet chamber 3 communicating, through the outlet conduit 32, with a mouthpiece applied thereto (not shown). The baffle 10 provides support for a spring 11 that compresses the head of the poppet 8 against the valve seat 5.

The regulator body also has a large opening 25 closed by a deformable diaphragm 13 that separates the outlet chamber 3 from the outside environment 12. Against a thin rigid plate 14 on the inner surface of the diaphragm 13, there rests the outer end 15 of a lever 16, the inner end 17 of which is hinged to the baffle 10 and attached to the tail 9 of the poppet 8 projecting from the baffle 10 into the outlet chamber 3. The mixture flows into the outlet chamber 3 through a passage 18. The end 17 of the lever 16 is shaped like a fork, as in the case of the previously-described known technique, see FIG. 4 in particular.

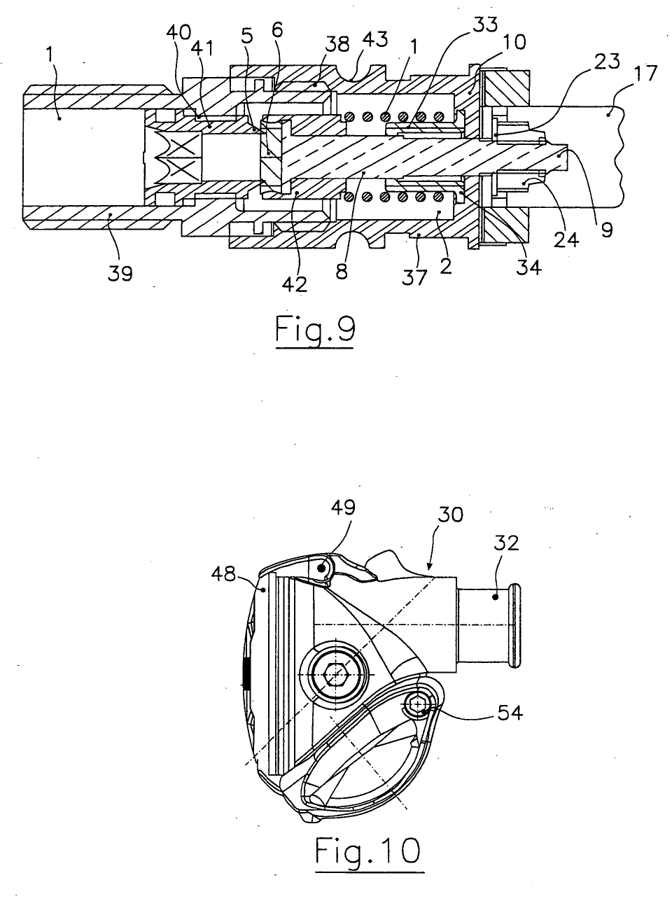

As also shown in greater detail in FIGS. 7 and 9, the annular opening between the hole 10a in the baffle 10 and the tail 9 of the poppet 8 is closed by a flexible sleeve 33, having a first, outer flange 34 facing the baffle 10, against which it is pressed by the spring 11 to form a seal inside a groove 10b. At its other end, the sleeve 33 has a second, inner flange 35 coupled in a circumferential groove 36 on the surface of the poppet 8 to form an airtight seal. The mixture can thus only pass from the intermediate chamber 2 to the outlet chamber 3 through the wide passage 18, preventing any blow-by, with its consequent cooling and freezing of the humidity contained in the escaping fraction of mixture, which would otherwise remain partly in the form of tiny ice crystals in the above-mentioned annular opening, creating friction against the tail of the poppet 8.

With reference to FIG. 9, the baffle 10 separating the intermediate chamber 2 from the outlet chamber 3 comprises the end of a first bush 37. At the end opposite the baffle 10, the bush 37 has an internal thread 38 coupled to the external thread of a second bush 39 that has an internal thread 40 in its medial region for screwing in a third bush 41, whose end facing the head 7 of the poppet 8 has an annular rib forming the valve seat 5 for engaging with the seal 6.

As a result, the third bush 41 forms the inlet chamber 1 inside the second bush 39, and the intermediate chamber 2 is formed between the third bush 41 and the baffle 10 of the first bush 37.

With reference to FIGS. 6 and 9, the head 7 of the poppet 8 has a ferrule 42 of rectangular cross section, whose section in the longitudinal plane shown in FIG. 6 has at least a partially-circular profile coming up against the inside wall of the intermediate chamber 2, which also has a substantially rectangular cross section, so that the whole poppet 8 can oscillate around a transversal axis C. To enable said poppet movement, the width of the ferrule, measured on the axis of oscillation, is narrower than the width of the intermediate chamber 2. Said oscillation enables the washer 23 (mounted so that it can slide on the tail 9 of the poppet 8) to move transversally in the direction of the arrows F3 (FIG. 7) together with the third flange 21 on the inner end 17 of the lever 16. There is consequently no sliding between the washer 23 and the two arms of the third flange 21 on the lever 16. This eliminates the second cause of friction, further reducing the vacuum that the user needs to generate when he inhales.

The assembly of this group of members in the second-stage regulator according to the present invention is as follows (FIGS. 6, 7, 9):

the flexible sleeve 33 is placed at the end of the first bush 37 and the spring 11 rests on its outer flange 34;

the ferrule 42 is fitted on the poppet 8 and the tail 9 of the poppet is then inserted through the spring 11, the sleeve 33 and the hole 10a in the baffle 10 forming the end of the first bush 37, in that order;

the washer 23 is inserted on the threaded end of the tail 9 of the poppet 8 and then the nut 24 is screwed into place;

the Z-shaped inner end 17 of the lever 16 is inserted between the washer 23 and the surface of the baffle 10 on the side facing the outlet chamber 3.

By adjusting the nut 24, the degree of tightness of the second bush 39, inside the first bush 37, and the degree of tightness of the third bush 41 inside the second bush, on the one hand it is possible to calibrate the force with which the seal 6 of the poppet 8 is pressed against the valve seat 5 and, on the other hand, by adjusting the degree of tightness of the nut 24 it is possible to calibrate the exact position of the end 15 of the lever 16.

The above-described assembly can be adjusted with the aid of a suitable tool before its installation in the regulator body 30 through the inlet conduit 31 on the regulator body. As shown in FIGS. 5 and 9, two opposite grooves 43 are formed into the outer surface of the first bush 37, perpendicular to the longitudinal axis of symmetry, and two holes 44, formed on the inlet conduit 31 at the same transversal distance from the grooves 43, are designed to contain two pins 45 when the aforesaid grooves are aligned with the holes 44. The relative longitudinal position of the assembly of the second-stage regulator vis-a-vis the inlet conduit 31 is thus perfectly defined. It is finally fixed in place by means of a nut 46 engaging the external thread on the second bush 39 until it abuts against the end of the inlet conduit 31.

As explained above, second-stage regulators of known type have a third source of friction due to sliding of the rounded outer end of the lever resting against the plate underneath the inner surface of the diaphragm. According to the invention, such sliding motion—and the consequent sliding friction—is converted into a rolling motion and the sliding friction is consequently replaced by a far more limited rolling friction.

For this purpose, the outer end 15 of the lever 16 has a profile such that it rolls along the underside of the plate 14, remaining at a tangent to the latter, as the diaphragm 13 and the plate 14 flex inwards from the resting position to the maximum expansion of the diaphragm. FIG. 8 shows a possible configuration of said end of the lever designed to operate as described above.

In practical terms, to achieve a rolling instead of a sliding motion between the lever and plate, it is necessary for the segment AB on the plate, coinciding with the set of points of contact between the lever and plate between the resting position and the maximum extension of the diaphragm, to coincide with the length of the arch A′B′ on the lever, and for the tangent in B′ to remain horizontal.

As illustrated in FIG. 5, the regulator body 30 is of elongated shape suitable for containing a diaphragm 13 that, according to the invention, takes on an elliptical shape. This solution enables the transversal dimension of the regulator to be kept within the overall dimensions of the surrounding apparatus, while nonetheless increasing the surface area of the diaphragm, with an evident benefit for the user, who saves energy because the vacuum he has to create by inhaling is lower the greater the surface area of the diaphragm. Moreover, the elliptical shape enables the plate 14 to remain parallel as it descends under the effect of a vacuum in the outlet chamber 3, a behavior that is fundamental to the proper operation of the lever and of the other moving parts in the regulator.

In second-stage regulators of known type, the diaphragm is attached to the edge of the corresponding opening by means of a covering frame generally screwed onto the regulator body after inserting an axially-movable control button, so that a slight pressure on said button enables the proper operation of the second-stage regulator to be checked.

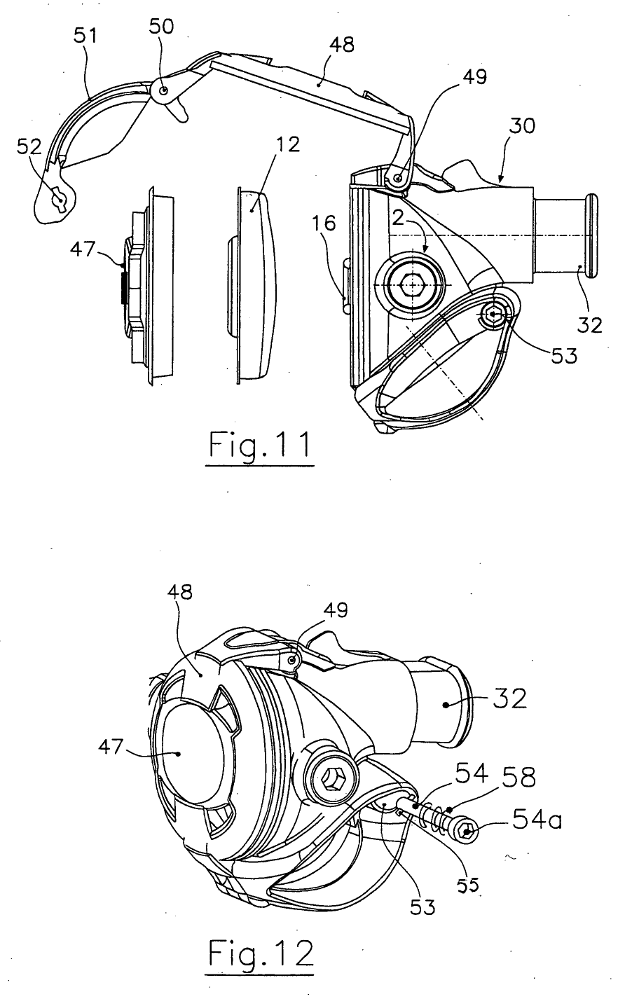

According to the present invention (FIGS. 10 and 11), the diaphragm 13 and the corresponding control button 47 are held against the edge of the corresponding opening in the regulator body 30 by means of a covering frame 48, one end of which is hinged at 49 to the regulator body 30, while the other end is hinged at 50 to a bracket 51 with a long through hole 52 suitable for aligning with a corresponding hole 53 in the regulator body, when the frame is in the closed position shown in FIG. 10. In this position, a pin 54 with an elongated head 55 is used to lock the assembly over the regulator body 30.

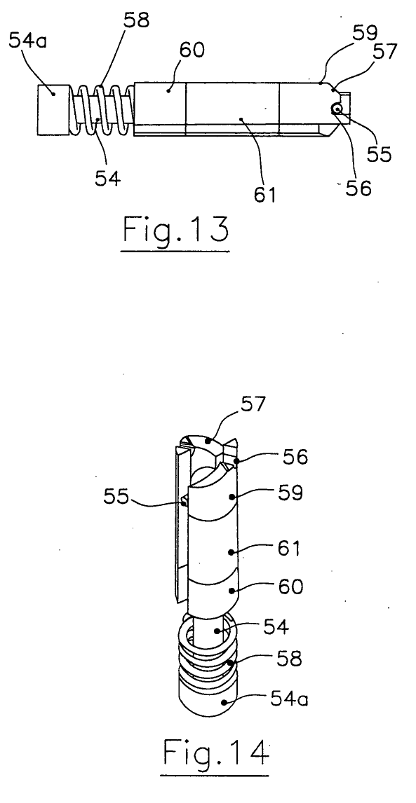

As shown in FIGS. 12, 13 and 14, the elongated head 55 on the pin 54 can take the form of a cross member engaging a seat 56 situated at the end of a cam profile 57. A spring 58 keeps the cross member 55 elastically in the closed position. FIGS. 13 and 14 show that the members 59 and 60 are integral to the regulator body 30, while the member 61 belongs to the end of the bracket 51. Any unwanted or accidental opening of the regulator due to the release of the bracket 51 and frame 48, and the consequent detachment of the diaphragm 13, is prevented by the fact that the pin 54 has a head 54a with a prism-shaped cavity designed so that a special key is needed to open it.

1 A second-stage regulator for scuba divers, which comprises a regulator body with an inlet conduit for connecting to a first-stage regulator that delivers a breathable gaseous mixture at a relatively constant pressure, an outlet conduit for connecting to a user's mouthpiece, and an opening blocked by a deformable diaphragm, the inlet conduit forming an inlet chamber and an intermediate chamber separated by a valve seat, against which a head of a poppet movable within the intermediate chamber is elastically pressed, a tail of the poppet projecting into an outlet chamber through an opening in a baffle that separates the intermediate chamber from the outlet chamber, and being connected to one end of a lever hinged to the baffle, the other end of the lever resting against the diaphragm so that the vacuum generated upon the user's inhalation results in an inward flexing of the diaphragm inside the outlet chamber and a rolling of the lever, with a consequent displacement of the poppet that, lifted away from the valve seat, allows for the passage of the gaseous mixture from the inlet chamber to the outlet chamber through the intermediate chamber and a passage from the intermediate chamber to the outlet chamber, wherein inside the intermediate chamber, coaxial to the poppet, a flexible sleeve is coupled by an airtight connection to the poppet and to the baffle around the opening.

2 The second-stage regulator set forth in claim 1, wherein between the baffle and the head of the poppet an elastic member is provided for urging the head against the valve seat, and wherein the flexible sleeve has, at one ends a first flange sealingly engaged with a corresponding groove on the inner surface of the poppet and, at the other ends a second flange urged by the elastic member against the baffle around the opening.

3 The second stage regulator set forth in claim 1, wherein the head of the poppet is set inside a ferrule of substantially rectangular cross section, whose section in the median longitudinal plane of the second-stage regulator, which also includes the lever, has at least an at least partially-circular profile abutting the inside wall of the intermediate chamber, enabling the poppet to oscillate in the longitudinal plane.

4 The second-stage regulator set forth in claim 3, wherein the intermediate chamber has a substantially rectangular cross section and the width of the ferrule measured along the axis of oscillation is generally less than the width of the intermediate chamber.

5 The second-stage regulator set forth in claim 1, wherein the end of the lever that is in contact with the diaphragm, or with a rigid plate attached to the diaphragm, has a generally arched shape with a profile such that the length of the arch between two adjacent points of contact measured along the lever is generally equal to the length of the segment between the same two adjacent points of contact measured along the diaphragm or the rigid plate.

6 A second-stage regulator for scuba divers, which comprises a regulator body with an inlet conduit for connecting to a first-stage regulator that delivers a breathable gaseous mixture at a relatively constant pressure, an outlet conduit for connecting to a user's mouthpiece, and an opening blocked by a deformable diaphragm, the inlet conduit forming an inlet chamber and an intermediate chamber separated by a valve seat, against which the head of a poppet movable within the intermediate chamber is elastically pressed, the tail of the poppet projecting into an outlet chamber through an opening in a baffle that separates the intermediate chamber from the outlet chamber, and being connected to one end of a lever hinged to the baffle, the other end of the lever resting against the diaphragm so that the vacuum generated upon the user's inhalation results in an inward flexing of the diaphragm inside the outlet chamber and a rolling of the lever, with a consequent displacement of the poppet that, lifted away from the valve seat, allows for passage of the gaseous mixture from the inlet chamber to the outlet chamber through the intermediate chamber and a passage from the intermediate chamber to the outlet chamber, wherein the head of the poppet is inside a ferrule of substantially rectangular cross section, whose section in a median longitudinal plane of the second-stage regulator, which also includes the lever, has at least an at least partially-circular profile abutting the inside wall of the intermediate chamber, enabling the poppet to oscillate in the longitudinal plane.

7 The second-stage regulator set forth in claim 6, wherein the intermediate chamber has a substantially rectangular cross section and the width of the ferrule, measured along the axis of oscillation, is generally less than that of the intermediate chamber.

8 A second-stage regulator for scuba divers, which comprises a regulator body with an inlet conduit for connecting to a first-stage regulator that delivers a breathable gaseous mixture at a relatively constant pressure, an outlet conduit for connecting to a user's mouthpiece, and an opening blocked by a deformable diaphragm, the inlet conduit forming an inlet chamber and an intermediate chamber separated by a valve seat, against which a head of a poppet movable within the intermediate chamber is elastically pressed, a tail of the poppet projecting into an outlet chamber through an opening in a baffle that separates the intermediate chamber from the outlet chamber, and being connected to one end of a lever hinged to the baffle, the other end of the lever resting against the diaphragm so that the vacuum generated upon the user's inhalation results in an inward flexing of the diaphragm inside the outlet chamber and a rolling of the lever, with a consequent displacement of the poppet that, lifted away from the valve seat, allows for passage of the gaseous mixture from the inlet chamber to the outlet chamber through the intermediate chamber and a passage from the intermediate chamber to the outlet chamber, wherein the end of the lever in contact with the diaphragm, has a generally arched shape with a profile such that the length of the arch between two adjacent points of contact measured along the lever is generally equal to the length of the segment between the same two adjacent points of contact measured along the diaphragm or the rigid plate.

9 The second-stage regulator set forth in claim 8, wherein the inlet conduit comprises a first bushing defining the intermediate chamber, the end of which forms the baffle, a second bush engaged with the first bush and defining the inlet chamber, and a third bush engaged with the second bush and defining the valve seat at one of its ends.

10 The second-stage regulator set forth in claim 9, wherein a member is provided for relative axial positioning of the first bush vis-à-vis the regulator body.

11 The second-stage regulator set forth in claim 10, wherein the axial positioning member comprises at least a pin for inserting in a hole formed in the regulator body suitable for fitting into a corresponding transverse groove in a side of the first bush.

12 The second-stage regulator set forth in claim 8, wherein the diaphragm is blocked inside the opening by a covering frame articulated to the regulator body, fasteners being provided for securing the frame to the regulator body.

13 The second-stage regulator set forth in claim 12, wherein a bracket fixable to the regulator body at its free end is hingedly connected to the frame.

14 The second-stage regulator set forth in claim 13, wherein a pin is provided for securing the bracket to the regulator body, the pin having a substantially T-shaped head passing through the free end of the bracket for snapping engagement with a seat in the regulator body after its rotation around its longitudinal axis, an elastic member coaxial to the pin being provided to prevent detachment of the substantially T-shaped head from the seat.
